(19) United States
(12) Patent Application Publication
(10) Pub. No.: US 2013/0072941 A1
(43) Pub. Date: Mar. 21, 2013

(54) Cement Injector and Cement Injector Connectors, and Bone Cement Injector Assembly

(71) Applicants: Francisca Tan-Malecki, West Lake Village, CA (US); Paul S. Maguire, Raynham, MA (US)

(72) Inventors: Francisca Tan-Malecki, West Lake Village, CA (US); Paul S. Maguire, Raynham, MA (US)

(21) Appl. No.: 13/561,705

(22) Filed: Jul. 30, 2012

Related U.S. Application Data
(60) Provisional application No. 61/535,794, filed on Sep. 16, 2011.

(51) Int. Cl. A61B 17/16

(52) U.S. Cl. 606/94

(57) ABSTRACT

An assembly for injecting bone cement into a vertebral body, comprising a bone cement dispenser, an introducer needle, a cement injection needle attached to the cement dispenser and slidably received in the introducer needle, and a Touhy-Borst adapter that locks and unlocks the cement injection needle from the introducer needle.

### CONTINUING DATA

This application claims priority from co-pending application U.S. Ser. No. 61/535,794, filed Sep. 16, 2011, entitled “Cement Injector and Cement Injector Connectors, and Bone Cement Injector Assembly” (Docket No. DEP6425USPSP), the specification of which is incorporated by reference in its entirety.

### BACKGROUND OF THE INVENTION

Vertebral body augmentation concerns the delivery of a curable bone cement into a fractured vertebral body. It is believed that, upon curing, the bone cement stabilizes the fracture site, thereby reducing micromotion and substantially eliminating pain. There are generally two types of vertebral body augmentation: vertebroplasty and kyphoplasty.

In vertebroplasty, the surgeon or clinician seeks to treat a compression fracture of a vertebral body by injecting bone cement such as PMMA into the fracture site. In one clinical report, Jensen, AJNR: 18 Nov. 1997, Jensen describes mixing two PMMA precursor components (one powder and one liquid) in a dish to produce a viscous bone cement; filling 10 ml syringes with this cement, injecting it into smaller 1 ml syringes, and finally delivering the mixture into the desired area of the vertebral body through needles attached to the smaller syringes.

Kyphoplasty is another form of vertebral body augmentation in which a cavity is first created within the fractured vertebral body (often by expanding and then withdrawing a balloon), after which cement is injected into the cavity.

Despite its success, vertebral body augmentation presents some challenges. For example, in vertebroplasty, the location of cement injection often starts anteriorly and moves posteriorly in the vertebral body by backing out the introducer needle from the vertebral body. With an osteoporotic patient, such movement of the introducer needle may cause a decrease in needle stability and anchorage within the vertebral body. Also in vertebroplasty, physicians encounter difficulty in quickly disconnecting and reconnecting the luer connection between cement reservoir and the introducer needle. Lastly, it has been found that cement tends to flow out of the cement reservoir when the reservoir is being screwed onto the luer connection on the introducer needle.

In balloon kyphoplasty, a 10 gauge introducer needle is not ideal to use when injecting cement directly into the cavity created in the vertebral body because the physician may waste a large volume of the cement if he/she does not clear the cannula during needle withdrawal. Also, for bipedicular access or multi-level procedures, the physician may not want to use multiple cement injector cannulae for cement injection. As with vertebroplasty, the physician practicing kyphoplasty may have difficulty in attaching a single cement reservoir to multiple cement injection needles. Lastly, one popular technique of cement injection uses prefilled bone filler devices that do not have a positive connection to the introducer needle. The bone filler device has a risk of sliding anteriorly towards the distal end of the created cavity and damaging the anterior vertebral body wall, which can provide a location for cement leakage.

In unthreading and threading the cement reservoir, physicians have noted without favor that cement continues to flow out of the distal end of the cement reservoir after injection. Because of this continued flow, it is difficult to properly attach the cement reservoir on the next introducer needle.

### SUMMARY OF THE INVENTION

The present inventors have developed solutions to the above-noted problems. These solutions center around the use of a Touhy-Borst adapter that releasably connects the introducer and cement injection needles.

Undesired movement of the introducer needle can be obviated through the use of a Touhy-Borst Adapter (TBA) between the cement injection and introducer needles. When the TBA connects the two needles, it prevents undesired movement between the cement injection needle and introducer needle. When the TBA is unlocked, it permits such movement.

In addition, cement reservoir-introducer connection issues can be solved by using a TBA that has a detachable connector.

Issues associated with bipedicular cement injection can be solved by using TBAs with detachable connectors that allow the quick transfer of a single injection needle from a first introducer needle to the next.

The risk associated with anterior sliding of a bone filler device may be obviated by the use of a detachable connector that can be locked, thereby prevent bone filler device movement.

Due to elimination of the unthreading and threading steps required in multi-level use, the connectors of the present invention provide the user with a quick method of transferring the cement reservoir for injection to multiple locations, which will provide the user with longer injection-capable periods during the cement's working time.

Using a “Touhy Borst”-like interface on the connector also desirably allows intraoperative adjustment of cannula length.

A small (<10 G) cement injector may be used to start cement injection anteriorly and move posteriorly in the vertebral body without moving the introducer needle and decrease needle stability.

Therefore, in accordance with the present invention, there is provided an assembly for delivering bone cement, the assembly comprising:
a) an introducer needle comprising:
   i) a hollow shaft having a sharpened distal end adapted for piercing bone and a proximal end portion forming a handle,
b) a connector attached to the handle of the introducer needle, the connector comprising:
   i) an annular base, and
   ii) a collet comprising a bore, wherein the collet is mounted on the annular base,
c) a cement injection needle comprising:
   i) a hollow shaft having a proximal end portion,
   ii) a handle formed on the proximal end portion of the hollow shaft thereof, and
   iii) a proximal connection for connecting to a cement reservoir, the connection extending from the proximal end portion of the hollow shaft of the cement injection needle,
      wherein the hollow shaft of the cement injection needle is slidably received in the bore of the collet.

### DESCRIPTION OF THE FIGURES

FIG. 1 discloses a cement injection needle mounted on a snap-fit connector.

FIG. 2 discloses a snap-fit connector of the present invention.

FIG. 3 discloses a snap-fit connector connected to both the cement injection needle and the introducer needle.

FIG. 4 discloses a cement injection needle mounted on a stud-clip connector.

FIG. 5 discloses a stud-clip connector of the present invention.

FIG. 6 discloses a stud-clip connector connected to both the cement injection needle and the introducer needle.

FIG. 7 discloses a cement injection needle mounted on a rotational lock connector.

FIG. 8 discloses a rotational lock connector of the present invention.

FIG. 9 discloses a rotational lock connector connected to both the cement injection needle and the introducer needle.

FIG. 10 discloses an assembly of the present invention connector to a bone cement injector.

### DETAILED DESCRIPTION OF THE INVENTION

Practicing the present invention can improve conventional vertebroplasty and balloon kyphoplasty procedures.

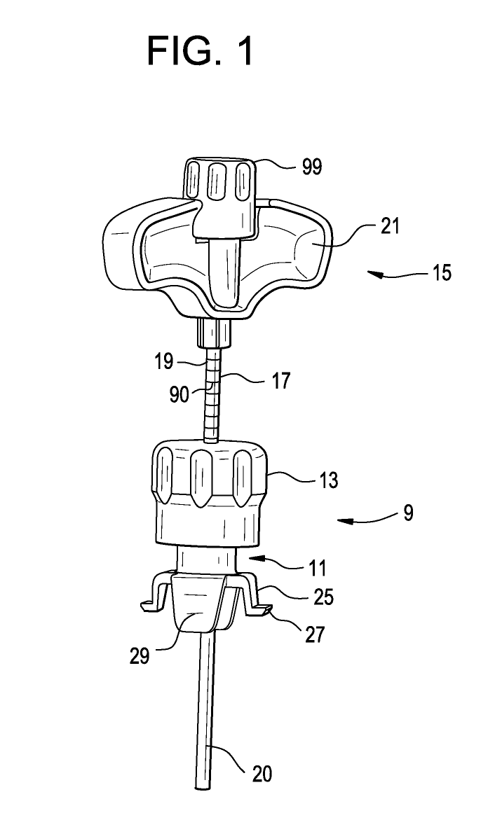

Now referring to FIGS. 1-3, there is provided an assembly for delivering bone cement, the assembly comprising:
a) an introducer needle 1 comprising:
   i) a hollow shaft 6 having a sharpened distal end (not shown) adapted for piercing bone and a proximal end 5 portion forming a handle 7,
b) a connector 9 attached to the handle of the introducer needle, the connector comprising:
   i) an annular base 11, and
   ii) a collet 13 comprising a bore, wherein the collet is mounted on the annular base,
c) a cement injection needle 15 comprising:
   i) a hollow shaft 17 having a proximal end portion 19 and a distal end portion 20,
   ii) a handle 21 formed on the proximal end portion of the hollow shaft thereof, and
   iii) a proximal connection (not shown) for fluidly connecting to a cement reservoir, the connection extending from the proximal end portion of the hollow shaft of the cement injection needle,
      wherein the hollow shaft of the cement injection needle is slidably received in the bore of the collet.

In some embodiments, a pair of arms 25 extend from the annular base of the connector. Preferably, each arm has a tip 27 extending radially from its respective arm, and each arm is flexible.

In some embodiments, the annular base of the connector comprises a pair of stabilization legs 29.

In some embodiments, the handle of the introducer needle comprises a channel 31 formed around the shaft of the introducer needle, wherein the connector is received in the channel.

The Touhy-Borst adapter (TBA) allows the user to move the distal location of the cement injector needle within the cavity without having to move the introducer needle. The TBA is well known in the medical arts and typically constitutes a rubber ring that tightens down on the shaft of the cement injector needle when an outer collet that surrounds the rubber ring is rotated clockwise. The outer collet provides for infinite, incremental adjustment of the amount of friction the rubber ring applies on the cement injector. Therefore, in the fully tightened/locked position, the TBA can prevent the cement injector from moving. The TBA can also provide adjustability in depth of the cement director with intentional tactile movement made by user. Lastly, the TBA can completely disconnect and provide friction-free movement of the cement injector in the open position.

Therefore, in accordance with the present invention, there is provided a method of stabilizing a spine, comprising the steps of:
a) inserting a first introducer needle into a first vertebral body,
b) inserting a cement injection needle having a Touhy-Borst adapter mounted thereon into the first introducer needle,
c) connecting the cement injection needle to the first introducer needle,
d) manipulating the Touhy-Borst adapter to adjust the cement injector to the desired injection depth within the vertebral body
e) flowing cement through the cement injection needle and into the first vertebral body.

Markings on the shaft of the cement injection needle provide a visual aid for the physician to accurately move the cement injection needle within the vertebral body. Laser-etched markings on the cement injection needle shaft will help the physician target the desired depth in the vertebral body. The external markings can help the physician make small depth adjustments without having to take a fluoroscopic image, thus reducing the amount of radiation exposure to the patient, physician and surgical staff.

By adding a connector in the form of a TBA between the introducer needle and cement injector needle, the user has more control of the flow of cement, thereby reducing risk to the patient. An attachable/detachable connection of the cement injector needle to the introducer needle handle allows the physician to use the cement injector needle at multiple locations and minimizes the need to reattach the cement reservoir onto multiple cement injector needles. The connector is mounted onto the cement injection needle and is preferably preset so that the distal end of the cement injection needle aligns with the distal end of the introducer needle when attached to the introducer needle. In the preset condition, the TBA is in its fully tightened/locked position to ensure that the cement injector needle does not move towards the anterior wall while being attached to the introducer needle. This reduces the risk of the cement injector needle sliding through the anterior wall and producing an avenue for cement leakage. From this preset position, the physician may unlock the TBA and move the distal end of the cement injection needle forward to a more desired location. Thereafter, the distal end of the cement injection needle may be incrementally refracted back to the preset position.

In some embodiments, the detachable connector comprises one of the following designs:
a) Snap-fit—a permanent attachment to the introducer needle;
b) Stud clip—a detachable attachment to the introducer needle. Application of force on the clip handles open and close the stud clip's connection arms for attachment and detachment,
c) Rotational lock—a detachable attachment to the introducer needle. It may be rotated clockwise to connect, and counter-clockwise to detach.

Each of these designs will now be discussed in more detail.

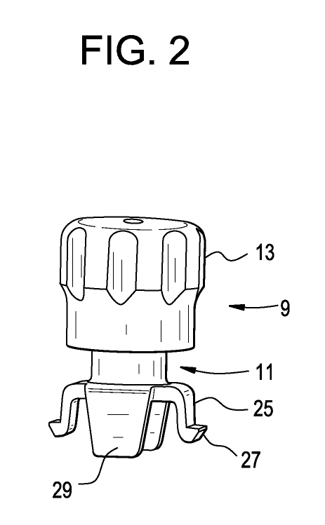

Now referring to FIGS. 1-2, there is provided a cement injection needle 21 locked to a snap-fit connector 9. The connector is preferably preset so that, when attached to the introducer needle, the distal end of the cement injection needle aligns with the distal end of the introducer needle. Marking lines 90 on the cement injection needle provide the clinician with visual guidance for injection depths in the vertebral body.

The snap-fit connector 9 comprises two main components. The top piece is the TBA 13 (which directly interfaces with the cement injection needle), while the bottom piece is the connector that directly attaches to the introducer needle. The connector comprises an annular ring 11 from which a pair of flexible arms 25 and a pair of stabilization legs 29 radially extend. The annular ring has a throughhole through which the cement injection needle may pass. The flexible arms have tips 27 at their ends that can lock into the introducer needle.

FIG. 1 further discloses a stylet comprising a proximal cap 99 and a distal shaft (not ahown). The stylet is located within the needle during insertion of the needle into the patient and acts to prevent tissue infiltration into the needle during insertion. After insertion, the stylet is removed from the needle.

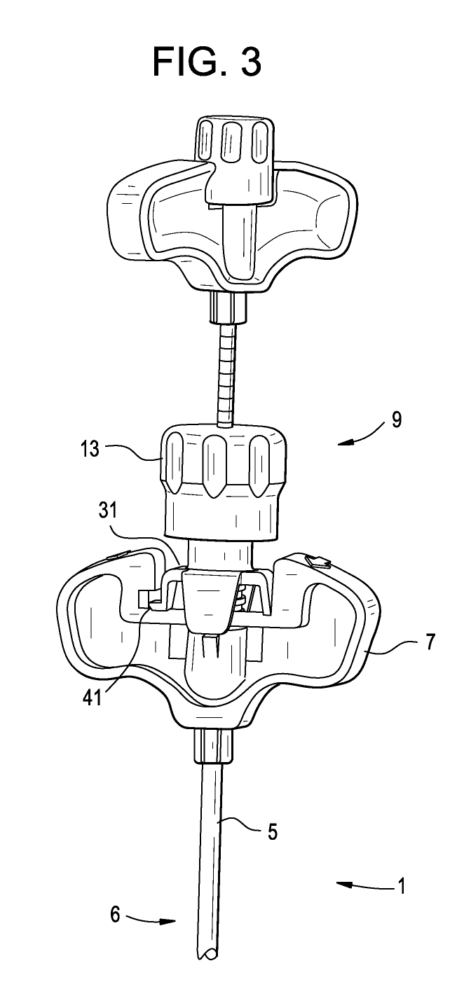

Now referring to FIG. 3, there is provided a snap-fit connector connected to both the cement injection needle and the introducer needle. In this FIG. 3, two arms on the snap-fit connector lock into pockets in the introducer needle. The stabilization legs on the snap-fit connector provide for stabilization and centering of the connector on the introducer needle. The introducer needle further comprises bilateral pockets that mate with the tips of the flexible arms of the snap-fit connector. In use, the arms are compressed by passage through channel 31 that opens onto the bilateral pockets. When the tips of the arms reach the pockets, the arms expand and thereby produce the snap-fit configuration.

In some embodiments, the channel comprises opposed pockets 41, and wherein the annular base of the connector comprises a pair of flexible arms 25 extending therefrom, wherein a portion of each arm is received in a respective pocket. In some embodiments, each arm has a tip 27 extending outwardly, wherein each tip is received in a respective pocket 41.

It is noteworthy that, although the snap-fit design embodiments succeed in enhancing control of the placement of the distal end of the cement injection needle, its connector is not detachable from the introducer needle. Accordingly, its use is limited to a single access point in the spine.

Therefore, it is a further object to provide embodiments that have the advantages of the snap-fit design that are also detachable, thereby allowing for its repeated use at different access points in the spine.

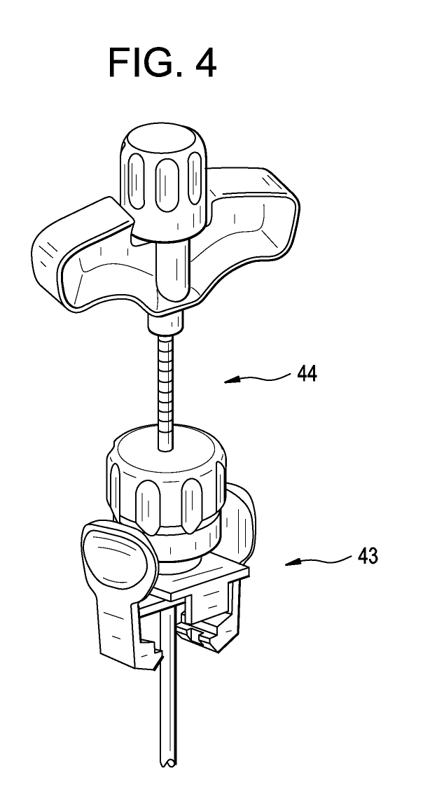

Now referring to FIG. 4, there is provided a stud clip connector of the present invention provided on a cement injection needle. The stud clip connector 43 is mounted onto the cement injection needle 44 and preset so that, when cement injection needle is attached to the introducer needle, the distal end of the cement injection needle aligns with the distal end of the introducer needle. Transverse marking lines upon the cement injection needle provide visual guidance for injection depths in the vertebral body.

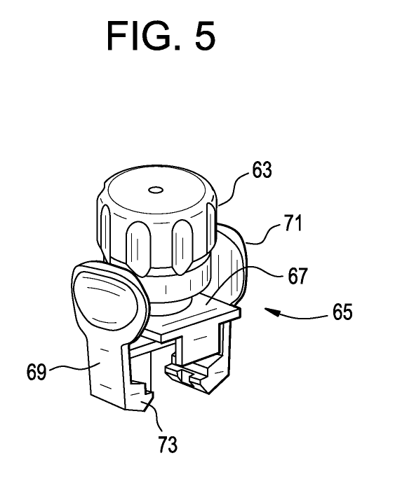

Now referring to FIG. 5, there is provided a stud clip connector by itself. Similar to the snap-fit connector, the stud clip connector 43 is made of two main components: a) a top piece that is a TBA 63 that directly interfaces with the cement injection needle, and b) a bottom piece 65 that is the connector that directly attaches to the introducer needle. The connector comprises a ledge 67 upon which the TBA seats and opposed connector arms 69 pivotally coupled to the ledge. The ledge has a throughhole (not shown) that allows for passage of the cement injection needle. On the proximal portion of each connector arm is a clip handle 71, while on the distal portion of each arm is a connecting tip 73. The clip handles provide a means of opening up the connector arms. The tips of the connector arms anchor the cement injection needle to the introducer needle.

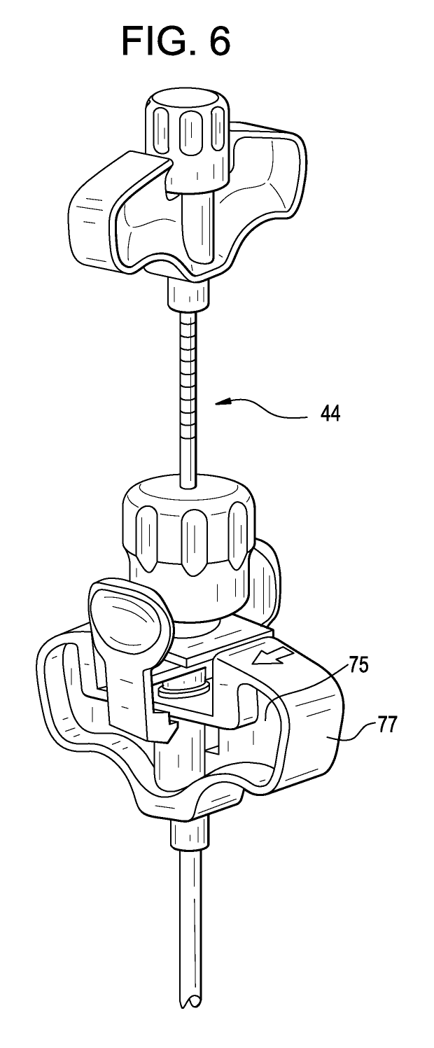

Now referring to FIG. 6, there is provided a stud clip connector connected to both a cement injection needle and an introducer needle. The tips of the connector arms grab the introducer needle under a bilateral ledge 75 provided on the opposed faces 77 of the introducer needle. Easy attachment and detachment of the connector to and from the cement injection needle can be accomplished by pinching the clip handles towards each other.

In some embodiments, each arm is pivotally connected to the annular base, wherein each arm is biased against the handle of the introducer needle, wherein each arm has a tip 73 extending inwardly, wherein each tip contacts the handle of the introducer needle.

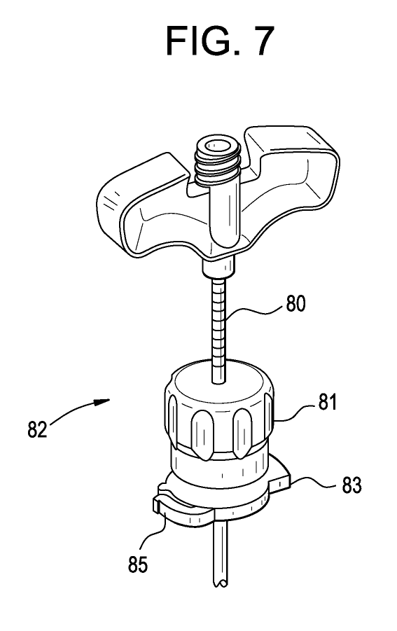

Now referring to FIG. 7, there is provided a cement injection needle 80 mounted onto a rotational lock connector 82. The connector is preset so that the distal end of the cement injection needle lines up with the distal end of the introducer needle when attached to the introducer needle. Transverse lines on the shaft of the cement injection needle provide the user with visual guidance for assessing injection depths in the vertebral body.

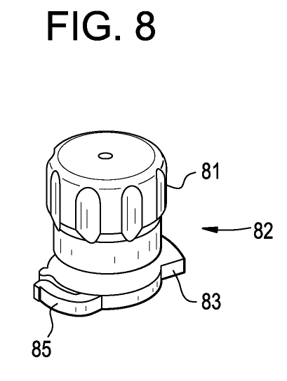

Now referring to FIG. 8, there is provided a rotational lock connector by itself. Similar to the above-discussed connector designs, the rotational lock connector is made of two main components. The top piece is the TBA 81 that directly interfaces with the cement injection needle. The bottom piece is the connector 83 that directly attaches to the introducer needle. The rotational lock provides a snap clip 85 that secures attachment of the cement injection needle to the introducer needle.

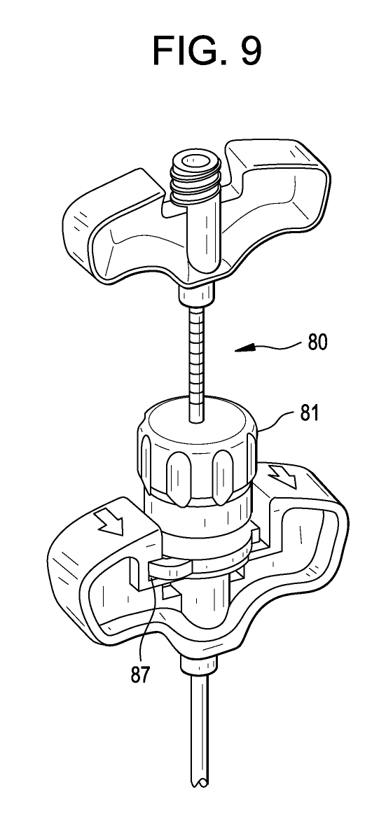

Now referring to FIG. 9, there is provided a rotational lock connector connected to both a cement injection needle and an introducer needle. The clip for the rotational lock connector snaps into an appropriate pocket 87 provided in the introducer needle handle. Attachment and detachment of the connector and cement injection needle can be done by rotating the connector. However, the TBA has a rotating feature as well and may hinder the ease of attachment and detachment.

For the stud clip and rotational lock designs, the cement injector can be used with more than one introducer needle and inject cement from multiple locations (i.e., from multiple levels or bilaterally). In such cases, the physician will need to detach the connector from one introducer needle and then attach the same connector to a second introducer needle, but will not need to unthread and thread the cement reservoir onto the introducer needles. The elimination of these unthread/thread steps saves the physician's time.

Additionally, it is believed that maintaining the connection between the cement injector needle and cement reservoir during introducer needle transfers improves the release of injection pressure when the hydraulic pump handle is turned counter-clockwise. In particular, the long length and small gauge of the cement injector needle yields significant friction and thereby helps reduce the flow of cement, thus mitigating loss of cement during the transfer of cement reservoir for injection to multiple locations.

In some embodiments, the assembly of the present invention is used in conjunction with a bone cement as disclosed in US Patent Publication 2010-0168271 (Beyar), the specification of which is hereby incorporated by reference in its entirety. In some embodiments, the assembly of the present invention is used in conjunction with a bone cement dispenser and cement, such as the CONFIDENCE™ SPINAL CEMENT SYSTEM, available from DePuy Spine, Inc. (Raynham, Mass.). In some embodiments, the assembly of the present invention is used in conjunction with a bone cement dispenser as disclosed in US Patent Publication 2010-0023017 (Beyar), the specification of which is hereby incorporated by reference in its entirety.

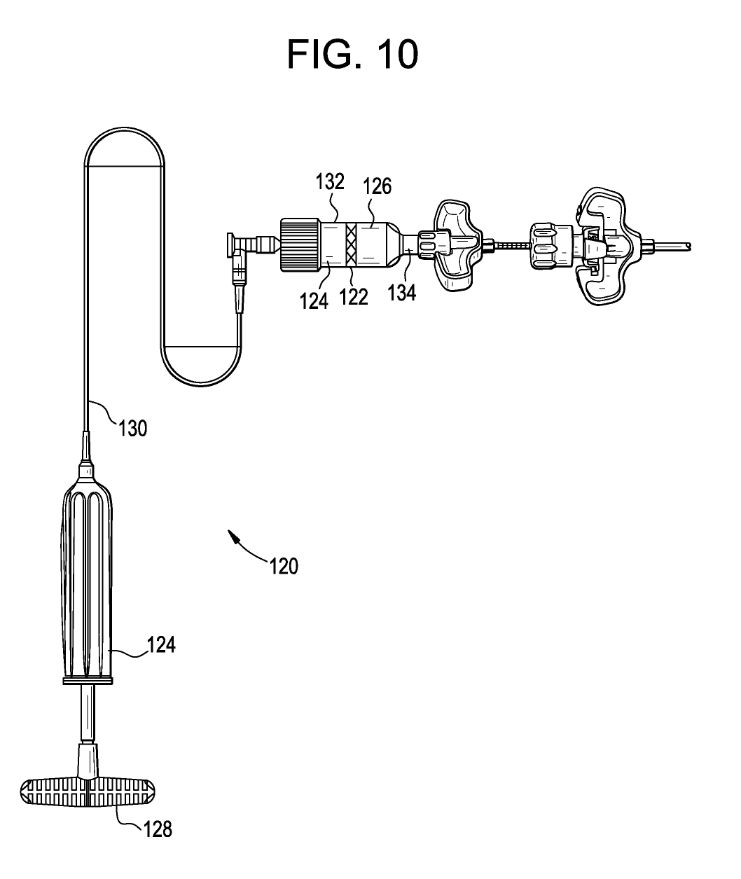

In one such embodiment, the handle of cement injector needle of the assembly of the present invention is fitted with a luer lock connection, and that connection is mated with the corresponding connection of a cement dispenser, as shown in FIG. 10. The bone cement dispenser 120 comprises a cement reservoir 132 connected by a saline-filled tubing 130 to pump. The reservoir has a piston 122 that separates the proximal saline 124 from the distal fluid cement 126. The pump 124 includes a distal saline reservoir in fluid connection with the tubing and a proximal piston that is actuated by a handle 128. In some embodiments, the cement reservoir has an ejection opening 134 forming a luer lock connector, wherein the luer lock connector of the cement reservoir is matingly attached to the luer lock connector of the cement injection needle. In preferred embodiments, the cement reservoir contains a flowable cement. Preferably, the flowable cement is acrylic-based.

In use, the physician first attaches the injection needle/TBA assembly to the bone cement dispenser. Next, the physician prepares the bone cement and fills the dispenser with the liquid cement. Next, the physician inserts the introducer needle into the targeted vertebral body, and then removes a stylet from the needle. The physician then inserts the injection needle/TBA assembly into the introducer needle and adjusts the TBA at the desired depth. The physician then actuates the dispenser to inject cement into the vertebral body. When filling is completed, the physician disconnects the TBA and removes the injection needle/TBA/cement dispenser assembly from the introducer needle.

Therefore, in accordance with the present invention, there is provided a method of stabilizing a vertebral body, comprising the steps of:
a) inserting a first introducer needle into a first side of the vertebral body,
b) inserting a cement injection needle into the first introducer needle,
c) flowing cement through the cement injection needle and into the first side of the vertebral body,
d) inserting a second introducer needle into a second side of the vertebral body,
e) withdrawing the cement injection needle from the first introducer needle,
f) inserting the cement injection needle into the second introducer needle, and
g) flowing cement through the cement injection needle and into the second side of the vertebral body.

Therefore, in accordance with the present invention, there is provided a method of stabilizing a spine, comprising the steps of:
a) inserting a first introducer needle into a first vertebral body,
b) inserting a cement injection needle into the first introducer needle,
c) flowing cement through the cement injection needle and into the first vertebral body,
d) inserting a second introducer needle into a second vertebral body,
e) withdrawing the cement injection needle from the first introducer needle,
f) inserting the cement injection needle into the second introducer needle, and
g) flowing cement through the cement injection needle and into the second vertebral body.

We claim:

1. An assembly for delivering bone cement, the assembly comprising:
a) an introducer needle comprising:
i) a hollow shaft having a sharpened distal end adapted for piercing bone and a proximal end portion forming a handle,
b) a connector attached to the handle of the introducer needle, the connector comprising:
i) an annular base, and
ii) a collet comprising a bore, wherein the collet is mounted on the annular base,
c) a cement injection needle comprising:
i) a hollow shaft having a proximal end portion,
ii) a handle formed on the proximal end portion of the hollow shaft thereof, and
iii) a proximal connection for connecting to a cement reservoir, the connection extending from the proximal end portion of the hollow shaft of the cement injection needle,
wherein the hollow shaft of the cement injection needle is slidably received in the bore of the collet.

2. The assembly of claim 1 wherein a pair of arms extend from the annular base of the connector.

3. The assembly of claim 2 wherein each arm has a tip extending radially from its respective arm.

4. The assembly of claim 3 wherein each arm is flexible.

5. The assembly of claim 2 wherein the annular base of the connector comprises a pair of stabilization legs.

6. The assembly of claim 1 wherein the handle of the introducer needle comprises a channel formed around the shaft of the introducer needle, wherein the connector is received in the channel.

7. The assembly of claim 6 wherein the channel comprises opposed pockets, and wherein the annular base of the connector comprises a pair of flexible arms extending therefrom, wherein a portion of each arm is received in a respective pocket.

8. The assembly of claim 7 wherein each arm has a tip extending outwardly, wherein each tip is received in a respective pocket.

9. The assembly of claim 1 wherein each arm is pivotally connected to the annular base, wherein each arm is biased against the handle of the introducer needle.

10. The assembly of claim 9 wherein each arm has a tip extending inwardly, wherein each tip contacts the handle of the introducer needle.

11. The assembly of claim 10 wherein each arm comprises a handle.

12. The assembly of claim 1 wherein the annular base comprises a snap clip adapted for rotational locking

13. The assembly of claim 12 wherein the annular base comprises a snap clip.

14. The assembly of claim 1 wherein the collet is adapted to close upon rotation.

15. The assembly of claim 1 wherein the collet is a Touhy-Borst adapter.

16. The assembly of claim 1, further comprising a cement reservoir having an ejection opening forming a luer lock connector, wherein the luer lock connector of the cement reservoir is matingly attached to the luer lock connector of the cement injection needle.

17. The assembly of claim 16 wherein the cement reservoir contains a flowable cement.

18. The assembly of claim 17 wherein the flowable cement is acrylic-based.

19. The assembly of claim 1 wherein the shaft of the cement injection needle comprises a plurality of regularly spaced, transverse marker lines for visual guidance.

20. The assembly of claim 1 wherein the proximal connection of the cement injector needle is a luer lock connection.

21. The assembly of claim 1 wherein the connector connects to the handle of the introducer needle by a snap fit connection.

22. The assembly of claim 1 wherein the connector connects to the handle of the introducer needle by a stud clip connection.

23. The assembly of claim 1 wherein the connector connects to the handle of the introducer needle by a rotational lock connection.

24. A method of stabilizing a vertebral body, comprising the steps of:
a) inserting a first introducer needle into a first side of the vertebral body,
b) inserting a cement injection needle into the first introducer needle,
c) flowing cement through the cement injection needle and into the first side of the vertebral body,
d) inserting a second introducer needle into a second side of the vertebral body,
e) withdrawing the cement injection needle from the first introducer needle,
f) inserting the cement injection needle into the second introducer needle, and
g) flowing bone cement through the cement injection needle and into the second side of the vertebral body.

25. The method of claim 21 wherein a connection between the cement injector needle and a cement reservoir containing the bone cement is maintained throughout steps c)-g).

26. A method of stabilizing a spine, comprising the steps of:
a) inserting a first introducer needle into a first vertebral body,
b) inserting a cement injection needle into the first introducer needle,
c) flowing cement through the cement injection needle and into the first vertebral body,
d) inserting a second introducer needle into a second vertebral body,
e) withdrawing the cement injection needle from the first introducer needle,
f) inserting the cement injection needle into the second introducer needle, and
g) flowing bone cement through the cement injection needle and into the second vertebral body.

27. The method of claim 23 wherein a connection between the cement injector needle and a cement reservoir containing the bone cement is maintained throughout steps c)-g).

28. A method of stabilizing a spine, comprising the steps of:
a) inserting a first introducer needle into a first vertebral body,
b) inserting a cement injection needle having a Touhy-Borst adaptoer mounted thereon into the first introducer needle,
c) connecting the cement injection needle to the first introducer needle,
d) manipulating the Touhy-Borst adapter to adjust the cement injector to the desired injection depth within the vertebral body
e) flowing cement through the cement injection needle and into the first vertebral body.
